(19) United States
(12) Patent Application Publication
(10) Pub. No.: US 2025/0006364 A1
(43) Pub. Date: Jan. 2, 2025

(54) Infant and Toddler Development Management System and Method by Artificial Intelligence-Based Video Analysis

(71) Applicant: YUHA-1, Daejeon (KR)

(72) Inventors: Miri Jeong, Seoul (KR); Byoung-Hee Han, Gwangmyeong-si (KR)

(21) Appl. No.: 18/217,375

(22) Filed: Jun. 30, 2023

(51) Int. Cl. G16H 50/20; A61B 5/00

(52) U.S. Cl. CPC G16H 50/20; A61B 5/1124; A61B 5/7264; A61B 5/1128

(57) ABSTRACT

A method comprises collecting questionnaires completed by guardians or test records filled out by experts for one or more developmental test items, selecting developmental test items that require imaging among developmental test items, providing an imaging guide for the selected developmental test items, capturing images of an infant or a toddler according to the imaging guide, pre-processing captured images and converting the pre-processed images into data suitable for AI analysis, a first diagnosis of analyzing converted data through AI to determine any presence of a developmental disorder and a developmental delay for selected developmental test items, a second diagnosis of determining any presence of a developmental disorder and a developmental delay for unselected developmental test items of the questionnaire or the test record, a comprehensive diagnosis of determining a comprehensive developmental disorder and developmental delay of an infant or a toddler, and providing a development report.

### TECHNICAL FIELD

The present disclosure relates to an infant and toddler development management system and method by artificial intelligence-based video analysis.

### BACKGROUND

Infant and toddler development tests are used to assess and understand the developmental stage of young children. These tests typically assess a child's developmental stage across various domains, including language, cognition, socio-emotional skill, and motor and help identifying any developmental disability or delay.

There are several test tools commonly used for infant and toddler development testing, examples of which include the Bayley scales of infant and toddler development screening test (Bayley-III), Denver Developmental Screening Test (DDST), Ages and Stages Questionnaires (ASQ).

These developmental screening tools are used by doctors, nurses, developmental psychologists, and educational professionals. Therefore, to have an infant and toddler developmental screening test using the developmental screening tool, a child needs to visit either a hospital or another specialized medical institution; in addition to an expert conducting the test, it is necessary for a guardian to complete a questionnaire. Since a lot of subjective factors are involved when the guardian fills out the questionnaire, relying on the guardian's questionnaire tends to diminish the reliability of the test.

### SUMMARY

To solve the problem above, the present disclosure provides an infant and toddler developmental screening method and system that are more accurate and may be performed without visiting a medical institution.

One aspect of the present disclosure provides an infant and toddler development management method through artificial intelligence (AI)-based image analysis.

The method comprises collecting questionnaires completed by guardians or test records filled out by experts for one or more developmental test items, selecting developmental test items that require imaging among developmental test items, wherein the developmental test items that require imaging include developmental test items that do not meet monthly age development milestones or developmental test items that do not follow a documentation guide, providing an imaging guide for the selected developmental test items, taking images of an infant or a toddler according to the imaging guide for the selected developmental test items, pre-processing the captured images and converting the pre-processed images into data suitable for AI analysis, a first diagnosis of analyzing the converted data through AI to determine any presence of a developmental disorder and a developmental delay for the selected developmental test items: a second diagnosis of determining any presence of a developmental disorder and a developmental delay for unselected developmental test items of the questionnaire or the test record, a comprehensive diagnosis of determining a comprehensive developmental disorder and developmental delay of an infant or a toddler based on the results of the first diagnosis and the second diagnosis, and providing a development report according to the comprehensive diagnosis result.

In one embodiment, developmental test items may include one or more of fine motor skill, gross motor skill, cognition, language, social skill, and self-help.

In one embodiment, the method may further comprise providing customized content and services according to a comprehensive diagnosis result.

In one embodiment, the method may be performed periodically to provide the development report together with time series information of the developmental test items.

In one embodiment, the providing of the imaging guide includes a calibration step of measuring body size and activity range of each child and modifying the imaging guide according to the measurements.

Another aspect of the present disclosure provides an infant and toddler development management system through artificial intelligence (AI)-based image analysis.

The system comprises a data collecting unit collecting questionnaires completed by guardians or test records filled out by experts for one or more developmental test items; a data selecting unit selecting developmental test items that do not meet monthly age development milestones or developmental test items that do not follow a documentation guide among developmental test items: an imaging guide providing unit providing an imaging guide for the selected developmental test items: an image capturing unit taking images of an infant or a toddler according to the imaging guide for the selected developmental test items: a pre-processing unit pre-processing the captured images and converting the pre-processed images into data suitable for AI analysis; and AI, Wherein the AI performs a first diagnosis of analyzing the pre-processed data to determine any presence of a developmental disorder and a developmental delay for the selected developmental test items and a second diagnosis of determining any presence of a developmental disorder and a developmental delay for unselected developmental test items of the questionnaire or the test record, determines a comprehensive developmental disorder and developmental delay of an infant or a toddler based on the results of the first diagnosis and the second diagnosis, and provides a development report.

In one embodiment, the AI may further provide customized content and services according to a diagnosis result.

In one embodiment, developmental test items may include one or more of fine motor skill, gross motor skill, cognition, language, social skill, and self-help.

In one embodiment, the AI may be lightweight using lightweight technology and may be installed on a mobile device.

In one embodiment, the lightweight technology may include weight pruning, weight sharing, and integer quantization techniques and transfer learning that transfers a pre-trained large model to a small model.

### BRIEF DESCRIPTION OF THE DRAWINGS

FIG. 1 illustrates an infant and toddler development assessment and management method through AI-based video analysis according to one embodiment of the present disclosure.

FIG. 2 is a flow diagram illustrating an infant and toddler development assessment and management method through AI-based video analysis according to one embodiment of the present disclosure.

FIG. 3 is an exemplary questionnaire that may be used by an infant and toddler development assessment and management method through AI-based video analysis according to one embodiment of the present disclosure.

FIG. 4 illustrates a calibration guide for precise recognition of an activity range according to one embodiment of the present disclosure.

FIG. 5 illustrates an exemplary imaging guide for gross motor skills that may be used by an infant and toddler development assessment and management method through AI-based video analysis according to one embodiment of the present disclosure.

FIG. 6 illustrates an exemplary imaging guide for fine motor skills that may be used by an infant and toddler development assessment and management method through AI-based video analysis according to one embodiment of the present disclosure.

FIGS. 7 and 8 illustrate exemplary AI motion label and motion models that may be used by an infant and toddler development assessment and management method through AI-based video analysis according to one embodiment of the present disclosure.

FIG. 9 illustrates a heatmap equation showing a process of detecting key features from image data utilizing image analysis techniques and generating a heatmap based on the intensity, color, or brightness of pixels or regions by AI according to one embodiment of the present disclosure.

FIG. 10 illustrates an inference method of an exemplary AI motion model that may be used by an infant and toddler development assessment and management method through AI-based video analysis according to one embodiment of the present disclosure.

FIG. 11 illustrates an example of a customized report through an infant and toddler development assessment and management method through AI-based video analysis according to one embodiment of the present disclosure.

FIG. 12 illustrates an example of a screen providing customized content through an infant and toddler development assessment and management method through AI-based video analysis according to one embodiment of the present disclosure.

FIG. 13 illustrates an example of a screen providing customized services through an infant and toddler development assessment and management method through AI-based video analysis according to one embodiment of the present disclosure.

### DETAILED DESCRIPTION

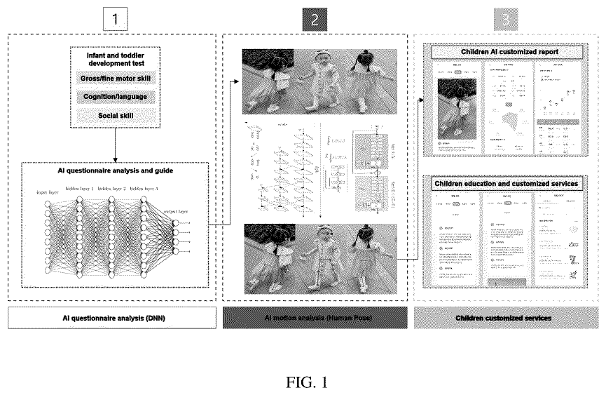

FIG. 1 illustrates an infant and toddler development assessment and management method through AI-based video analysis according to one embodiment of the present disclosure. Detailed descriptions will be further provided below.

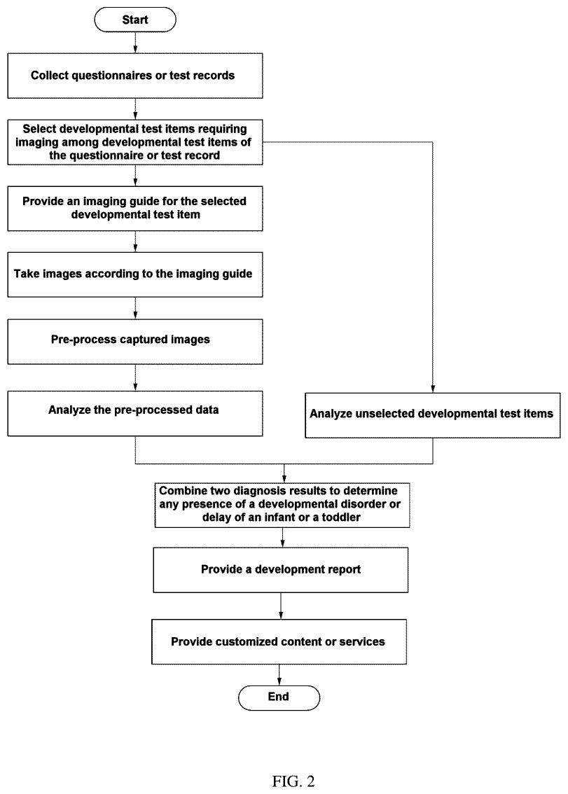

FIG. 2 is a flow diagram illustrating an infant and toddler development assessment and management method through AI-based video analysis according to one embodiment of the present disclosure.

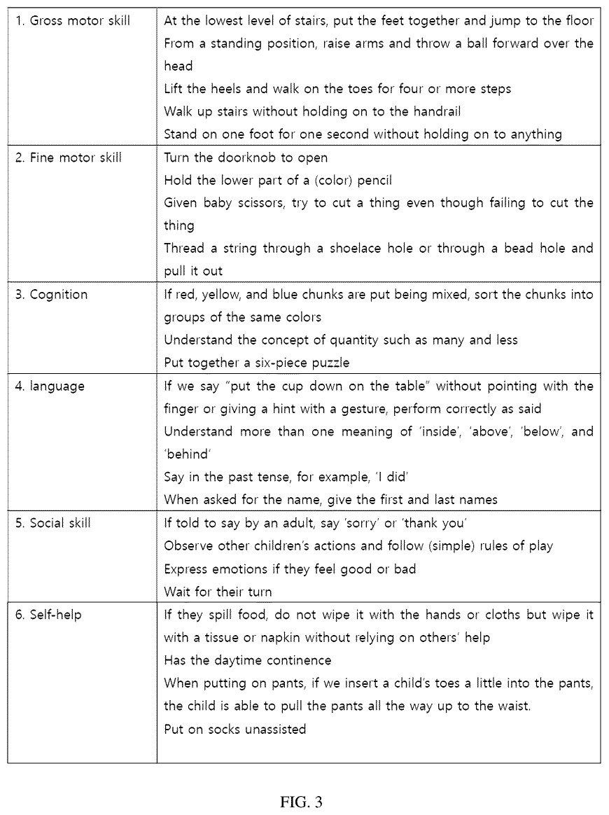

As shown in the figure, the method begins with collecting questionnaires or test records. In the collecting step, questionnaires completed by guardians or test records filled out by experts are collected. For example, a developmental screening questionnaire may be collected from a guardian of an infant or a toddler to be assessed through a mobile or web service. These questionnaires or test records include various developmental test items. For example, the developmental test items may include one or more of fine motor skill, gross motor skill, cognition, language, social skill, and self-help. FIG. 3 shows an exemplary questionnaire.

Next, the method proceeds to the step of selecting developmental test items that require imaging among the developmental test items of a questionnaire or a test record. For example, among the developmental test items, a developmental test item that does not meet the monthly age development milestones or a developmental test item that does not follow a documentation guide may be selected as a developmental test item that require further imaging.

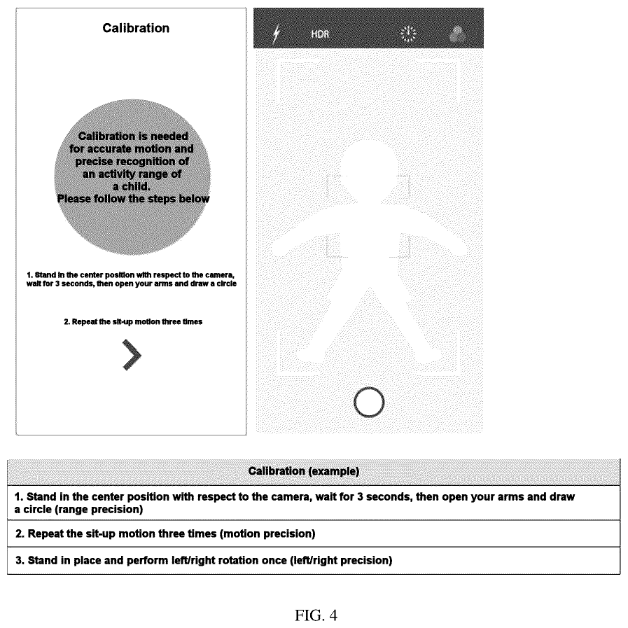

Then, the method proceeds to the step of providing an imaging guide for a selected developmental test item to a guardian of an infant or a toddler. For example, an imaging guide for a motion, including the measurement of the front of the whole body or measurement of the side of the whole body and a screen composition, may be provided in the form of motion graphics for each selected developmental test item. Also, since the body size or proportion, and activity range of children have separate characteristics for each age and individual, a calibration guide for accurate motion and precise recognition of the activity range for each child may be provided. FIG. 4 illustrates a calibration guide for precise recognition of an activity range according to one embodiment of the present disclosure. An infant performs specific motions at the center position with respect to a camera, by which an AI model may improve the range precision, motion precision, and left and right precision.

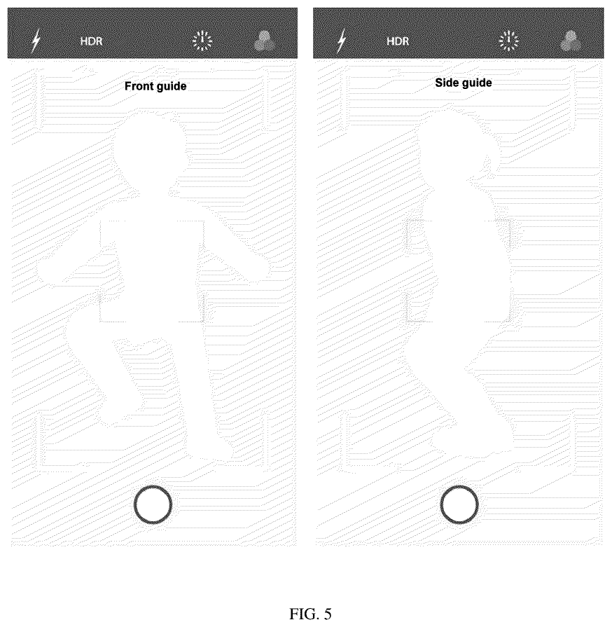

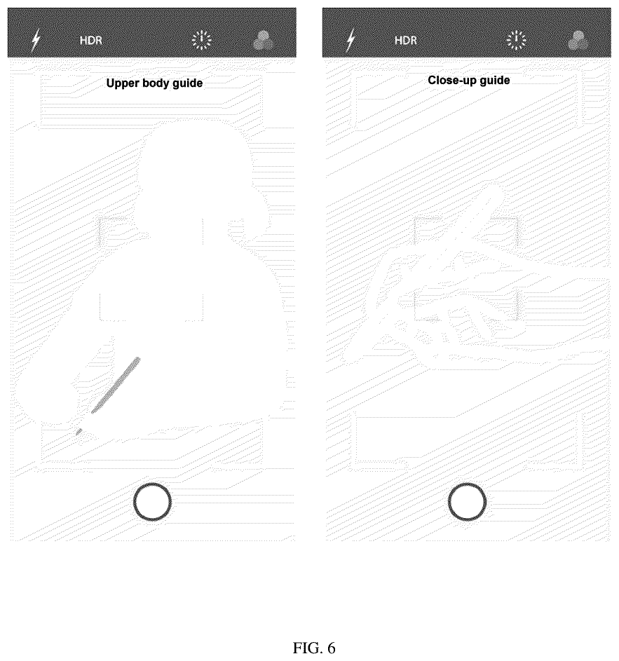

FIG. 5 illustrates an exemplary imaging guide for gross motor skills that may be used by an infant and toddler development assessment and management method through AI-based video analysis according to one embodiment of the present disclosure. The method provides an imaging guide for the front and side according to a motion, which is suitable for a gross motor developmental test item. FIG. 6 illustrates an exemplary imaging guide for fine motor skills according to one embodiment of the present disclosure. The method provides an imaging guide for the hand, face, upper body, and the like according to specific parts of fine muscles, which is helpful for recognizing a body part relevant to a fine motor developmental test item.

Next, the method proceeds to the step of imaging according to an imaging guide. In this step, images of a motion of an infant or a toddler are captured according to the imaging guide for a selected developmental test item.

Then, the method proceeds to the step of pre-processing the captured images. In the pre-processing step, the captured image data is pre-processed to be suitable for AI-based analysis. Techniques used for the pre-processing include resizing to adjust the size of the image, normalization to adjust pixel values within a specific range, color conversion to change colors or to convert a color image to a grayscale image, background removal, noise removal, region of interest (ROI) extraction, and data augmentation.

Then, it proceeds to the step of analyzing the pre-processed data. In the analyzing step, AI analyzes the pre-processed data to determine an infant's developmental stage and developmental delay for each selected developmental test item. For example, the analyzing step may compare pre-processed data for each developmental test item with a predetermined threshold value and determines a developmental delay if the threshold value is not reached. Also, the analyzing step may compare the pre-processed data for each developmental test item with a developmental stage table to determine a corresponding developmental stage.

Separately from the above step, analyzing unselected developmental test items is performed. In this step, the AI analyzes the unselected developmental test items and identifies an infant's developmental stage and developmental delay for each unselected developmental test item. For example, data for each developmental test item may be compared with a predetermined threshold value, and a developmental delay may be diagnosed if the threshold value is not reached. In addition, the pre-processed data for each developmental test item may be compared with a developmental stage table to identify a corresponding developmental stage. This step may be performed concurrently with the analyzing of the selected developmental test items or may be performed, for example, while imaging is conducted for efficient use of computational resources.

Next, the method collects the results of the respective developmental test items and proceeds to comprehensively diagnose an infant's integrated development stage and developmental delay. For example, this step may determine a developmental stage or developmental delay by scoring a result of each developmental test item and comparing the score with a predetermined threshold value.

Then, the method proceeds to the step of providing a development report. This step provides a development report containing the results analyzed through AI. FIG. 11 illustrates an example of a development report through an infant and toddler development assessment and management method through AI-based video analysis according to one embodiment of the present disclosure. As shown in the figure, a development report includes an assessed monthly age for each item, a comparison result with an average value, an integrated developmental level, and a graph visualizing the above. Also, by performing this method periodically, for example, monthly, quarterly, or at a predetermined specific monthly age, the development report may provide time series information for each item.

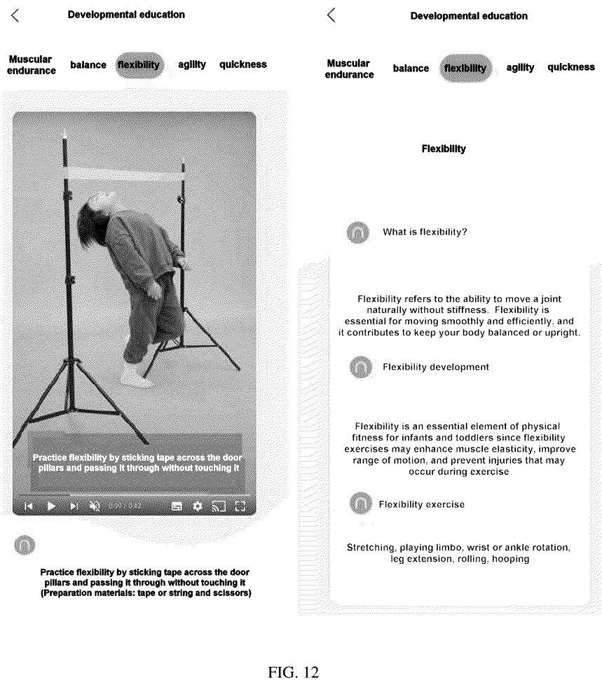

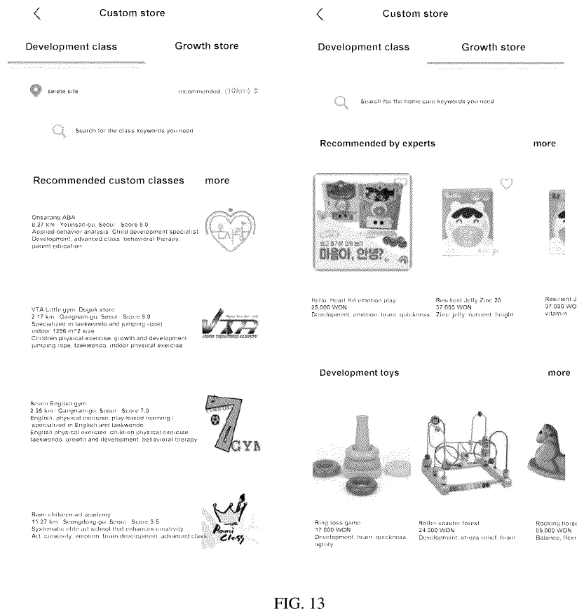

As an option, the method proceeds to the step of providing customized content and services. This step provides play, exercise, and educational content for each stage of growth and development. For example, this step provides customized commerce, educational programs, exercise and education centers, and hospitals and clinics. FIGS. 12 and 13 illustrate examples of a screen providing customized content or services through an infant and toddler development assessment and management method through AI-based video analysis according to one embodiment of the present disclosure.

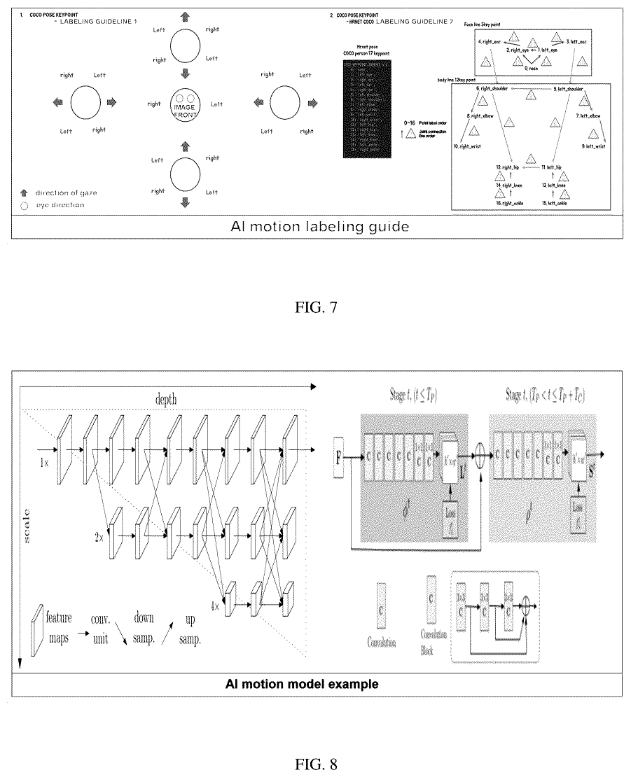

FIGS. 7 and 8 illustrate exemplary AI motion label and motion models that may be used by an infant and toddler development assessment and management method through AI-based video analysis according to one embodiment of the present disclosure. In the illustrated example, the AI model is a joint-based HRNET human pose model composed of 15 to 17 key points ((x, y)-2D image coordinates) based on the Common Objects in Context (COCO) dataset, the Max Planck Institute for Informatics (MPII) dataset, and the Crowd-Pose dataset. This AI model conducts labeling by considering left and right imaging of the video/image and assigns a value even to a hidden joint labeling area through visibility.

The AI motion model shown in FIG. 8 is built on a multi-layered structure composed of a starting layer, a merging layer, and a combining layer and is thus capable of inferring both small parts and the overall structure required for estimating a child's pose, thereby improving the accuracy of behavior analysis of children. Also, this model utilizes high resolution to extract features at various levels of image resolution: a typical network structure starts with low resolution and extracts features at different resolution step by step. Also, a technique of partitioning an input image into sub-images of different resolutions and extracting features from each partitioned region may be used. Multi-scale fusion may be performed to combine features of different resolutions effectively, and a technique of maintaining high-resolution information by interconnecting features extracted from various resolutions may be utilized.

In another embodiment, different AI models such as the OpenPose, AlphaPose, Spatial Temporal Graph Convolutional Network (ST-GCN), and Long Short-Term Memory (LSTM) may be employed.

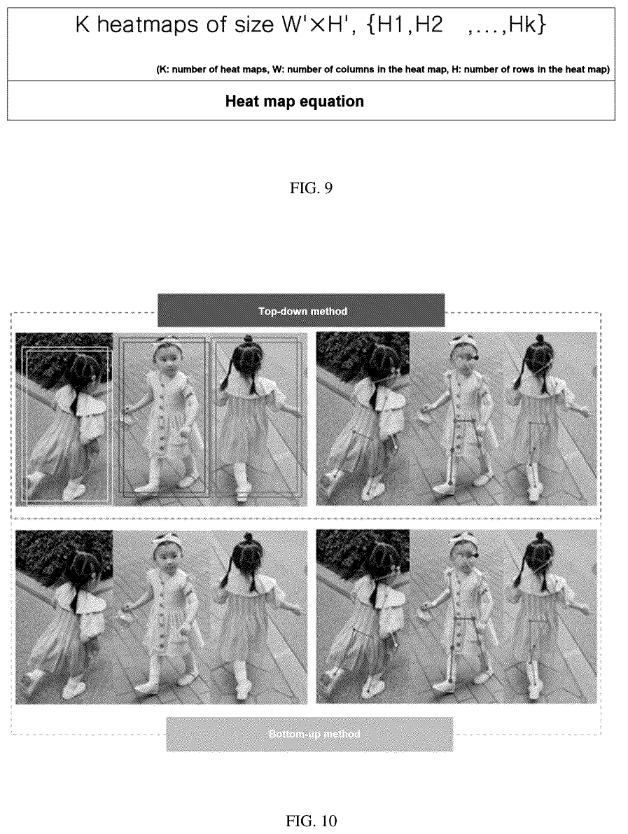

FIG. 9 illustrates a heatmap equation showing a process of detecting key features from image data utilizing image analysis techniques and generating a heatmap based on the intensity, color, or brightness of pixels or regions by AI according to one embodiment of the present disclosure. FIG. 9 is a heat map visualization process that visualizes the importance of each point in color or brightness through the generated heat map. Through the heat map, a user may easily recognize the joint position, activity level, and motion pattern. The joint positions are determined by a high-resolution heat map (“K heat maps of size W×H, {H1, H2, . . . , Hk}, where each heat map consists of W columns and H rows).

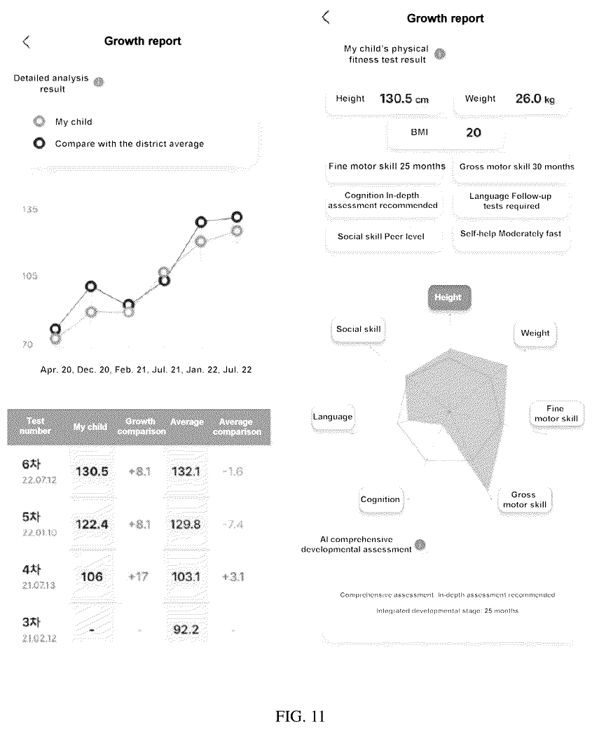

FIG. 10 illustrates an inference method of an exemplary AI motion model that may be used by an infant and toddler development assessment and management method through AI-based video analysis according to one embodiment of the present disclosure. In the illustrated example, the reasoning method of the model supports top-down and bottom-up methods. The top-down method may improve the inference accuracy by estimating the joints of a secondary cognition child model from a primary human cognition model: the bottom-up method may reduce the inference time by analyzing only the motion. Both types of inference methods may be used interchangeably in the system.

These AI models may be lightweight and agile using lightweight technology, such as weight pruning, weight sharing, integer quantization techniques, and transfer learning that transfers a large pre-trained model to a small model, which may thus be mounted on a mobile device.

Although preferred embodiments of the present disclosure have been described above, it should be clearly understood by those skilled in the art that the embodiments are provided only as illustrative examples. Various modifications, changes, and substitutions may be made by those skilled in the art without departing from the technical principles and scope of the present disclosure. It should be understood that various alternatives of the embodiments of the present disclosure may be implemented in arbitrary combinations. The scope of the appended claims defines the scope of the present disclosure and attempts to include the methods and structures that fall within the scope of the equivalents of the embodiments of the present disclosure.

What is claimed is:

1. A method comprising:
receiving questionnaires or test records associated with one or more developmental test items,
selecting developmental test items that require imaging among the one or more developmental test items, wherein the one or more developmental test items that require imaging include developmental test items below a monthly age development milestones threshold or developmental test items that do not follow a documentation guide,
providing an imaging guide for the selected developmental test items,
capturing images of an infant or a toddler according to the imaging guide for selected developmental test items,
pre-processing captured images and converting the pre-processed images into data suitable for artificial intelligence (AI) analysis,
performing, by an AI, a first diagnosis of analyzing the converted data for a presence of one or more developmental disorders and developmental delays for the selected developmental test items,
determining, by the AI, a second diagnosis of a presence of the one or more developmental disorders and the developmental delays for unselected developmental test items of the questionnaire or the test record,
providing a comprehensive diagnosis based on the determined presence of the one or more developmental disorders and the developmental delays of an infant or a toddler based on results corresponding to the first diagnosis and the second diagnosis, and
providing a development report based on the comprehensive diagnosis.

2. The method of claim 1, wherein the developmental test items include one or more of fine motor skill, gross motor skill, cognition, language, social skill, and self-help.

3. The method of claim 1, further comprising:
providing customized content and services according to the comprehensive diagnosis.

4. The method of claim 1, wherein the method is performed periodically to provide the development report together with time series information of the developmental test items.

5. The method of claim 1, wherein the providing of the imaging guide includes a calibration step of measuring body size and an activity range of each child and modifying the imaging guide according to the measurements.

6. A system comprising:
a data collecting unit configured to collect questionnaires completed by guardians or test records filled out by experts for one or more developmental test items;
a data selecting unit configured to select developmental test items below a monthly age development milestones threshold or developmental test items that do not follow a documentation guide;
a guide providing unit configured to provide an imaging guide for the selected developmental test items;
an image capturing unit configured to capture images of an infant or a toddler according to an imaging guide for the selected developmental test items;
a pre-processing unit pre-processing the captured images and converting the pre-processed images into data suitable for artificial intelligence (AI) analysis; and
an AI, wherein the AI is configured to:
perform a first diagnosis of analyzing the pre-processed data to determine any presence of one or more developmental disorders and developmental delays for the selected developmental test items and a second diagnosis of determining any presence of the one or more developmental disorders and the developmental delays for unselected developmental test items of the questionnaire or the test record,
determine a comprehensive developmental disorder and developmental delay of an infant or a toddler based on the results of the first diagnosis and the second diagnosis, and
provide a development report corresponding to the comprehensive development disorder.

7. The system of claim 6, wherein the AI further provides customized content and services according to the comprehensive diagnosis.

8. The system of claim 6, wherein developmental test items include one or more of fine motor skill, gross motor skill, cognition, language, social skill, and self-help.

9. The system of claim 6, wherein the AI is a below a weight threshold using lightweight technology and is installed on a mobile device.

10. The system of claim 9, wherein the lightweight technology includes weight pruning, weight sharing, and integer quantization techniques and transfer learning.
